(19) United States
(12) Patent Application Publication
(10) Pub. No.: US 2005/0130162 A1
(43) Pub. Date: Jun. 16, 2005

(54) Diagnostic and therapeutic tools for the x-linked mental retardation syndrome

(75) Inventors: Ilaria Meloni, Siena (IT); Alessandra Renieri, Siena (IT)

Correspondence Address: Albert Wai Kit Chan;Law Offices of Albert Wai Kit Chan, World Plaza Suite 604, 141 07 20th Avenue, Whitestone, NY, 11357, US

(73) Assignee: Universita degli Studi di Siena, 55-53100 Siena (IT)

(21) Appl. No.: 10/507,145

(22) PCT Filed: Mar. 6, 2003
(86) PCT No.: PCT/IT03/00134

(30) Foreign Application Priority Data
Mar. 8, 2002 (IT) RM2002000130

(51) Int. Cl. C12Q 1/68; C07H 21/04; C12P 19/34

(52) U.S. Cl. 435/6; 435/91.2; 536/23.2

(57) ABSTRACT

A nucleic acid comprising at least one fragment of the human FACL4 gene or FACL4 protein or functional portions thereof for diagnostic or therapeutic purposes applied to syndromes associated with mental retardation is described. Appropriate diagnostic kits are also described.

The present invention concerns diagnostic and therapeutic tools for X-linked mental retardation syndrome.

X-linked mental retardation (XLMR) is an inherited condition in which the inability to develop cognitive processes is caused by mutations of one gene on the X chromosome. In a recent review of XLMR case reports, 136 cases of “syndromic” or “specific” MR (MRXS) and 66 cases of “nonspecific” MR (MRX) were grouped together (1). In only 9 of the 66 cases of MRX was the responsible gene identified (1). Hence, other specific genes that may be responsible for mental retardation need to be identified for both diagnostic and therapeutic purposes.

The recent discovery of contiguous genes deletion in ATS-MR syndrome (Alport syndrome and mental retardation) has led to the identification of region Xq22.3 as containing a gene for mental retardation (2).

The authors of the invention performed comparative analysis on the extension of the deletion in patients affected by ATS-MR and in those with ATS alone. The comparison led the authors of the invention to restrict the potentially critical region for mental retardation to approximately 380 kb, containing at least four genes. The authors have now identified three point mutations, two missense mutations and a mutation that induces a change in the splice site in the FACL4 gene in three families with nonspecific MR. An analysis of the enzymatic activity in the lymphoblastoid cell lines of three patients showed a marked reduction in enzymatic activity compared with normal cells, demonstrating that all three mutations were destructive. All female carriers with point mutations in the FACL4 gene or genomic deletions showed completely skewed X-inactivation, suggesting a role of the gene in conferring a selective advantage. FACL4 is the tenth mutated gene associated with MRX (1) and the first to be involved in the metabolism of fatty acids.

The objective of the present invention is a nucleic acid molecule comprising at least one fragment of the human FACL4 gene that encodes for a functional portion of the FACL4 protein to be used in the diagnosis of MR-associated syndromes.

A further object of the invention is a nucleic acid molecule comprising at least one fragment of the human FACL4 gene that encodes for a functional portion of the FACL4 protein to be used in the therapy of MR-associated syndromes.

It is in the scope of the invention a method to detect in a subject at least one mutation of the gene encoding for the human FACL4 protein, located on the X chromosome, comprising the phases of:

a) collecting a specimen containing a sufficient quantity of the subject's DNA or able to be reproduced in culture;

b) isolating the DNA of said sample;

c) exponentially amplifying the DNA using as a primer pair for amplification reaction at least two oligonucleotides able to amplify a fragment of the human FACL4 gene, in which the fragment encodes for a functional portion of FACL4 protein;

d) demonstrating in at least one amplified fragment any mutations compared with a healthy control.

Preferably, the exponential DNA amplification phase will be performed using primer pairs for the amplification reaction able to amplify the entire coding portion of the human FACL4 gene. More preferably, the exponential DNA amplification phase to amplify the entire coding portion of the human FACL4 gene will comprise the use of the following primer pairs:

| | |
|---|---|
| Exon 3: | 5′ GTGAGCACATTTAGCTTAAG 3′, |
| | 5′ ATCAATTGTGCTATCAACTTG 3′; |
| Exons 3 and 4: | 5′ CTTCTTCAGCACAATAAGGC 3′, |
| | 5′ GCATACTTAAAACGCACTCG 3′; |
| Exon 5: | 5′ CCGCTCATAGCTTCTGTATG 3′, |
| | 5′ AACAATTCTCACATGCAAGC 3′; |
| Exons 6 and 7: | 5′ AGACTGACTTCAATAATATCC 3′, |
| | 5′ TCATTTGTTTCCCTAACCTAC 3′; |
| Exon 8: | 5′ ATTGATAGCTTATCGTTATGC 3′, |
| | 5′ AATGCTGAACATGAACTCTG 3′; |
| Exon 9: | 5′ ATGATAAAGCTCTTGGTATTTC 3′, |
| | 5′ TGCAGCATCATACGATCATG 3′; |
| Exon 10: | 5′ AATTCCAAGTGTAACTTCTG 3′, |
| | 5′ TAAAAGGTCCAAGTACGATC 3′; |
| Exon 11: | 5′ ACTGTCTCCATTCCTTTCAG 3′, |
| | 5′ ACCTTATGATCATGGTGGTG 3′; |
| Exon 12: | 5′ GAGGAATCTTTCCCAGAGC 3′, |
| | 5′ ATTAGTAGCAGCTGATACAG 3′; |
| Exon 13: | 5′ TATTCCCAGTGCATTGGTAC 3′, |
| | 5′ GAAAGTCATAAAGCTGACAG 3′; |
| Exon 14: | 5′ CTAATGTTCTCTCATAAAGTG 3′, |
| | 5′ GAACTAATGGAACCATCAAC 3′; |
| Exon 15: | 5′ CAGTCAGAATTGCATATACC 3′, |
| | 5′ AAGAGAAGACTATGTTACCC 3′; |
| Exon 16: | 5′ TTGGAATTATCTGTACTGTAC 3′, |
| | 5′ AGCCTAATGCAAAAGACATC 3′; |
| Exon 17: | 5′ ACTCCTTTCTCGTCTCTTTC 3′, |
| | 5′ TAGAGGTTGAAAACCACCAG 3′. |

In a preferred embodiment, the phase of demonstrating, in at least one amplified fragment, mutations compared with a healthy control will be done by direct sequencing or the SSCP method.

A further object of the invention is a diagnostic kit for MR-associated syndromes, using the method described above, and comprising:

a) at least one pair of primer oligonucleotides for the exponential amplification reaction of at least one fragment of the human FACL4 gene, in which the fragment encodes for a functional portion of the FACL4 protein;

b) a control DNA from a subject not affected by XLMR.

Preferably, the oligonucleotide primer pairs for the amplification reaction are able to amplify the entire region encoding for the FACL4 gene.

A further object of the invention is the FACL4 protein or a functional portion thereof for the diagnosis of MR-associated syndromes.

A further object of the invention is the FACL4 protein or a functional portion thereof for the therapy of MR-associated syndromes.

Within the scope of the invention is a method for the determination of the enzymatic activity of FACL4 protein in a biologic sample, comprising the phases of:

a) collecting a biological sample from the subject, in which the sample is comprised in the group of biologic fluids, lysated lymphoblastoid cells, leukocytes;

b) incubating the sample in an appropriate reaction mixture containing arachidonic acid;

c) detecting arachidonyl-CoA production.

In a preferred form, the detection of arachidonyl-CoA is performed using labeled arachidonic acid, alternatively with chromotographic methods, such as HPLC, or spectrophotometry.

A further object of the invention is a diagnostic kit for MR-associated syndromes, using the method described above, and comprising:

a) lysis buffer, with appropriate protease inhibitors and/or reduction agents;

b) Coenzyme A and Adenosine 5′triphosphate (ATP);

c) Cold arachidonic acid and ¹⁴C-labeled arachidonic acid.

The invention is described below in reference to its explicative but not limitative embodiments, in reference to the following figures:

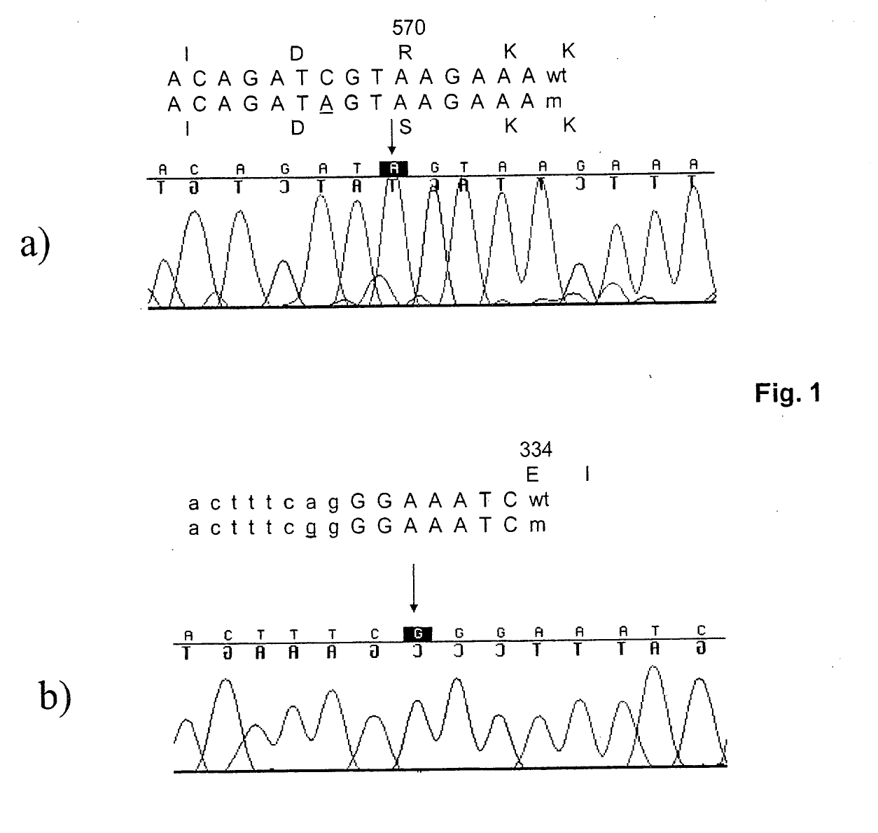

FIG. 1—Mutated Sequence Chromatograms.

a) Mutation in exon 15 of proband T22 in family MRX63; b) mutation in intron 10 of proband P55. The chromatograms refer to the antisense helix. The nucleotide and amino acid change are shown above the chromatograms. The intron bases are indicated in lower case letters. Wt=wild type sequence; m=mutated sequence.

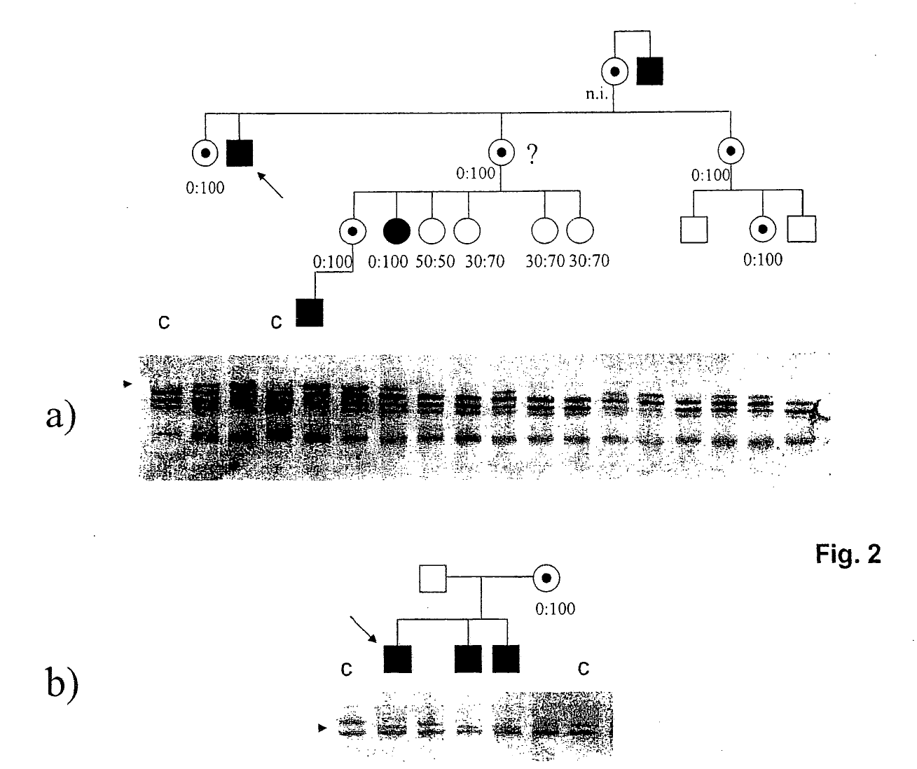

FIG. 2—Segregation Analysis.

An SSCP analysis with the GenePhor apparatus (Pharmacia-Biotech) is shown. The pedigrees are illustrated above the lines. An arrow indicates the propositus of each family. The numbers below the symbols indicate the percentage of X-inactivation. C=Control. The question mark in the upper pedigree near female 11.3 refers to an uncertain phenotype of this female. Her current low IQ could derive at least partly from a different disease that associates MR with ataxia (Table 1). An arrowhead indicates the mutated conformer.

a) PCR product of exon 15 of family MRX63. Primers 5′CAGTCAGAATTGCATATACC3′ and 5′AAGAGAAGACTATGTTACCC3′ were used.

b) PCR product of exon 11 and flanking intron sequences from the family of proband P55. Primers 5′ACTGTCTCCATTCCTTTCAG3′ and 5′ACCTTATGATCATGGTGGTG3′ were used.

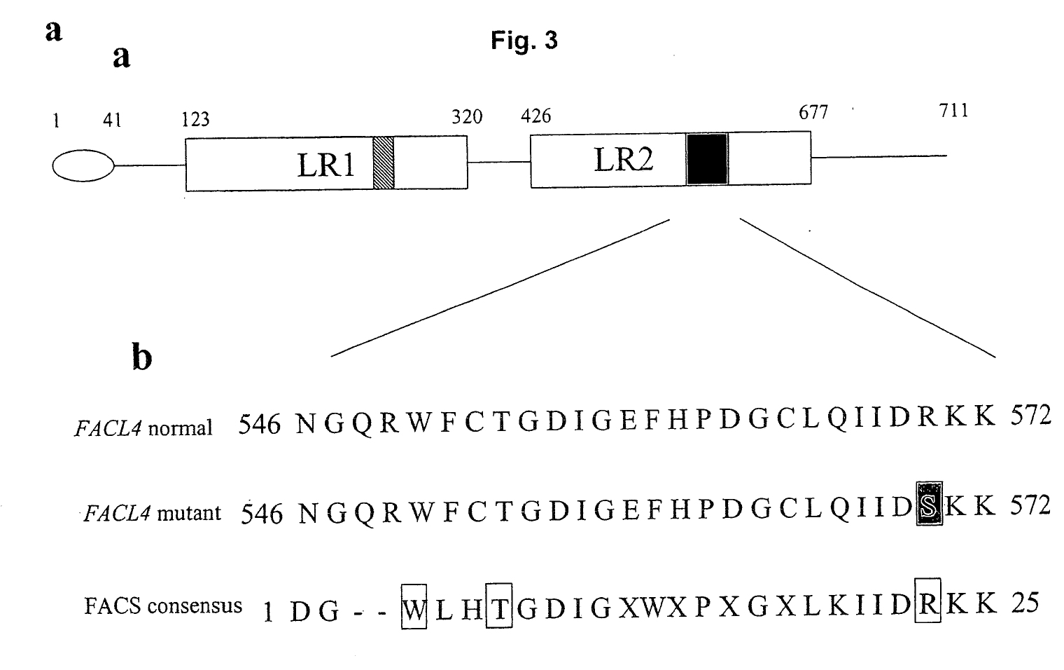

FIG. 3—Schematic Representation of Protein FACL4 (a) and Comparison of Motifs Characterizing acyl-Coenzyme A Synthetases (FACS) (b).

a) The Neuro-specific N-terminal peptide (ellipse) is followed by two luciferase domains (LR1 and LR2) containing the AMP-binding domain (striped box) and the FACS signature motif (gray box), respectively;

b) The normal and mutated sequences are aligned with the consensus sequence (25 amino acids long) of the FACS. The mutated amino acid in family MRX63 is shown in gray. The three amino acids whose substitution causes the loss of enzymatic activity in acyl-CoA synthetase of E. Coli are boxed (9).

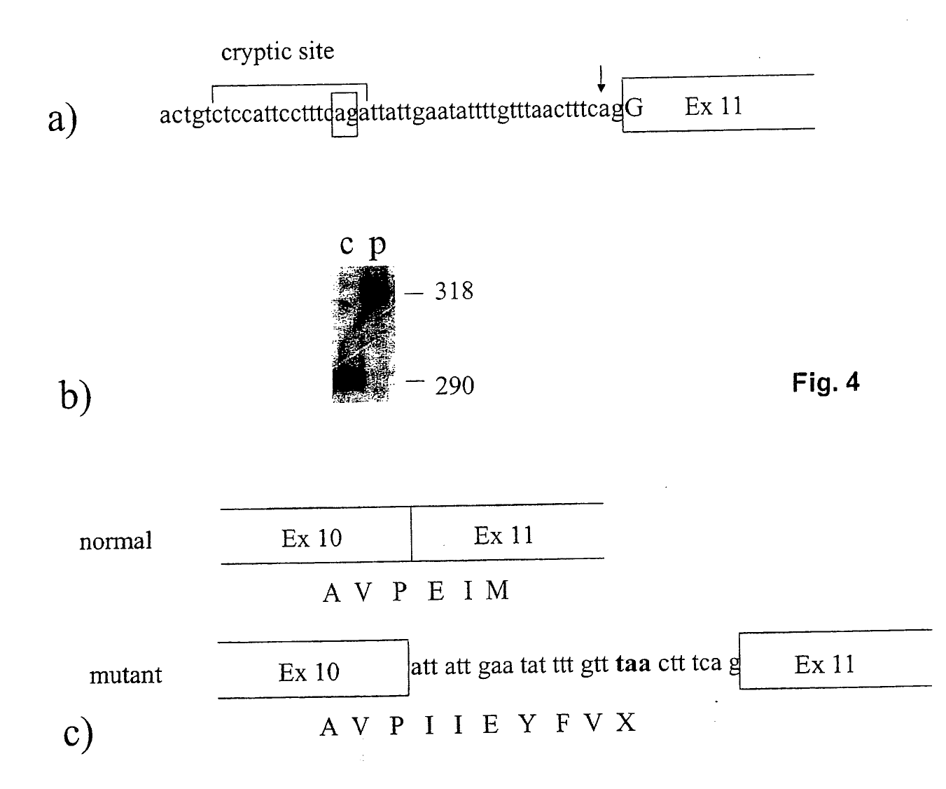

FIG. 4—RT-PCR on Proband P55.

a) Normal sequence around intron 1 0--exon 11 junction. Note the cryptic splice site. An arrow indicates the site of mutation;

b) 6% polyacrylamide gel of the RT-PCR product from exon 10 to exon 12.

The expected RT-PCR product of 290 bp shifted to 318 bp in the mutated RNA. C=control; p=patient.

c) Normal and mutated RNA. The amino acids are indicated below the nucleotides. In the mutated RNA there is a stop codon (TAA, indicated in bold) after 6 abnormal codons deriving from intron sequences.

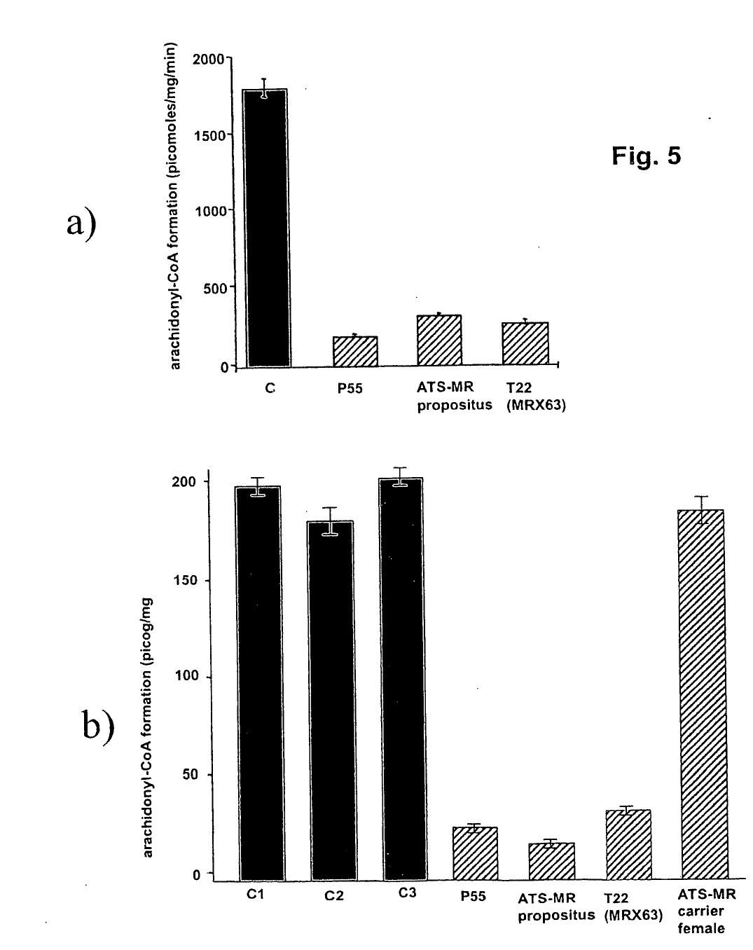

FIG. 5—Activity of arachidonyl-CoA Synthetase of Mutants and Controls.

Assays for arachidonyl-CoA synthetase activity on whole cell lysates from 10⁸ lymphoblastoid cells of 3 normal controls (C1-C3), patients P55 and T22, patient ATS-MR and his mother (female carrier with genomic deletion). The graphs show the mean activity from assays performed in triplicate. The results are representative of three independent experiments. Statistical evaluation between groups was performed using Student's t test (P=0.001).

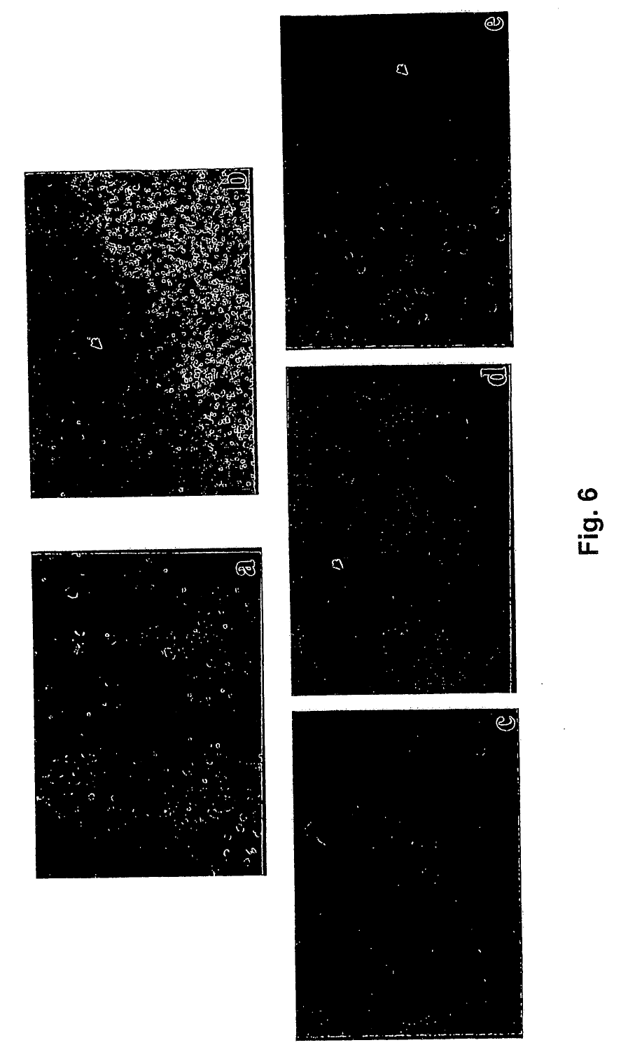

FIG. 6—Expression of FACL4 in the Human Hippocampus and Cerebellum.

Staining with immunoperoxidase (light brown). Counterstaining with Mayer's hematoxylin (violet).

a) Section of the hippocampus (x640). Cells of the dentate gyrus (left, enlarged in c) and pyramidal cells (right, enlarged in d) showing strong cytoplasmic immunoreactivity.

b) Section of the cerebellum (x640). Reactivity of Purkinje's cells and cells of the granular layer; in the molecular layer there are sparse immunoreactive cells and weakly reactive elongated processes (distal part of Purkinje's cells [arrowhead]).

c) Higher magnification of the dentate gyrus (x1500).

d) Pyramidal cells of the hippocampus with dense cytoplasmic staining (x1500). Nonreactive nuclei are clearly delineated by a denser ring (arrowhead).

e) Higher magnification of the same region of the cerebellar section in b (x1500) showing that cytoplasmic staining continues from the cell soma to the dendrites of Purkinje's cells (arrowhead). The nucleus is surrounded by a thin intensely reactive ring.

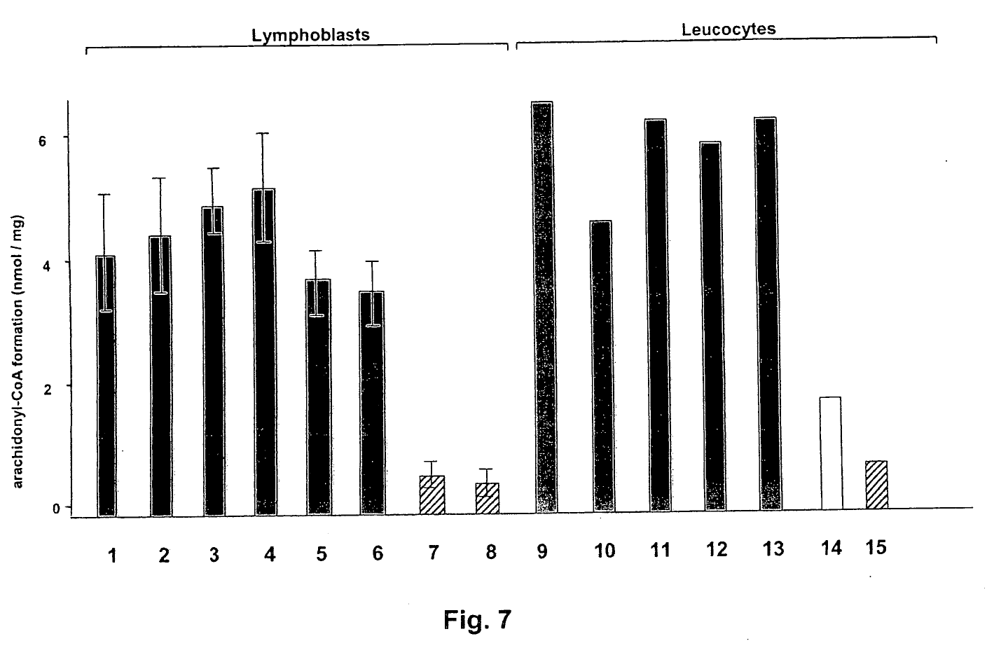

FIG. 7—Arachidonyl-CoA Synthetase Activity in Leukocytes.

The assay was carried out on whole cell lysates of 10⁷ lymphoblastoid cell lines (columns 1-8) and of leukocytes isolated from 10 ml of blood samples (columns 9-15). For lymphoblastoid cell lines, results show the means of at least three independent experiments. Statistical evaluation between groups was done with Student's t-test (p=0.01).

Column 1=normal control; 2=L22; 3=K8045; 4=K8435; 5=K8835; 6=K8610; 7=L46 (MRX68); 8=male patient with genomic deletion (ATS-MR syndrome); 9=L49; 10=L56; 11=control from 10 ml of blood after cryo-preservation. 12-14=controls after 24h, 72h, and 120h of blood preservation at room temperature. 15=blood of L46 (MRX68) stored at room temperature for 24 hours. Columns 7, 8 and 15 (stripped box) and column 14 (white box) were significantly different from controls.

### METHODS

Physical Examination and Psychometric Assessment of Family MRX63

An accurate and complete physical examination was performed on patients I1, I2, II2, II3, II4, III1, III2, III4, IV1. No healthy male could be examined. The psychometric assessment was performed for 9 subjects from this family, including 2 affected males (I2,II2) and 7 females (carriers: I1, II3, II4, III1, III2 and normal homozygotes III3, III4). The Columbia Mental Maturity Scale (18) was used to determine IQ. In addition, specific skills were assessed from selected tests such as expressive language (19), verbal memory (Digit Span of the McCarthy Scales for Children's Abilities [MSCA]) (20), spatial memory (21), and visuo-spatial organization (Copying geometrical shapes, MSCA). Some executive functions were also evaluated, such as verbal fluency (MSCA), visual selectiveness (22) and impulsiveness-resistance (Luria's test). Mental retardation was diagnosed in the presence of all of the following criteria: significantly sub-average intellectual functioning (IQ</=70) (criterion A), significant limitations in adaptive functioning (criterion B) and onset of the disorder before 18 years of age (criterion C) (23). Female 1114 has a low IQ (56) but did not meet the other criteria and so was not considered “affected”. Subject IV1, a child, was not examined by a neuropsychologist, but previous assessments indicated severe-to-moderate mental retardation, with an IQ of 37 (Brunet-Lezine).

Isolation of RNA and RT-PCR

The procedure with TRIZOL (Life Technologies) was used to isolate RNA from EBV-transformed lymphoblasts from proband P55 and control individuals. cDNA synthesis was carried out in a reaction volume of 20 μl with total RNA (1-2 μg), specific primers (5′ATGMTCGGTGTGTCTGAGG3′, 5′ATCCCATGGAGATGTTCTGTC3′) (1 μM each), dNTP (2 mM), RNAse inhibitors (5U) and MMLV-RT (25U) (Advanced Biotechnologies LTD, Epsom, UK). Primers and RNA were pre-incubated at 70° C. for 5 min and then the other reagents were added and the reaction was then incubated at 42° C. for 60 min and at 75° C. for 10 min. The cDNAs were amplified with specific primers on exon 10 (5′GGMGCAAAGGAACTGTAC3′) and on exon 12 (5′ ATGAATCGGTGTGTCTGAGG 3′).

Mutation Analysis of Patients

Direct sequencing was performed on 12 families mapping to large regions of the X chromosome, comprising Xq22.3 (from Xq2l to Xq26), collected by the European XLMR Consortium: 3 from Nijmegen (N9, N32, N50), 3 from Paris (P4, P14, P15), 5 from Tours (T11, T18=MRX66, T19, T22=MRX63, T40) and 1 from Leuven (L17). Direct sequencing was performed in both directions with the BigDye Terminator Cycle Sequencing kit (Applied Biosystems) on an ABI 310 Automated Sequencer; sequences were analyzed using the Genescan package software. In addition, 107 families whose X-linked inheritance was established on the basis of pedigree analysis were examined: 45 from Nijmegen, 41 from Paris, 9 from Leuven and 12 from Berlin. For all families, other causes of MR were excluded (normal phenotype, normal neurological findings, normal karyotype, normal metabolic screening, exclusion of FRAXA by molecular studies with FMR1). All were classified as highly suggestive of X-linked inheritance based on the presence of at least two affected males and of maternal transmission. In addition, the patients of all families were examined by members of the XLMR consortium. Mutation analysis in these 107 families was performed using the Single Strand Conformation Polymorphism (SSCP) technique (29). For this technique, the PCR products of coding exons of FACL4 (from 3 to 17) were denatured and electrophoresed on 6% polyacrylamide gel or on a 6-12.5% gradient (GeneGelExcel Kit, Pharmacia); the DNA was then revealed with silver staining. The technique is based on the principle that an alteration in the nucleotide sequence causes an altered migration of single-stranded DNA, which then yields a different pattern compared with unaltered DNA. Also, direct sequencing was performed on 4 additional families from Leuven, 2 with a diagnosis of nonspecific XLMR (L22 and L46) and 2 with a diagnosis of X-linked spastic paraplegia (L49 and L56). All four families mapped to large regions of the X chromosome encompassing Xq22.3 (from Xp11.4 to Xq26.1). The coding exons of FACL4 (from 3 to 17), comprising both alternative start codons located in exons 3 and 4 (5), were amplified with specific primers. The primers were:

| | |
|---|---|
| Exon 3: | 5′ GTGAGCACATTTAGCTTAAG 3′ |
| | 5′ ATCAATTGTGCTATCAACTTG 3′ |
| Exons 3 and 4: | 5′ CTTCTTCAGCACAATAAGGC 3′ |
| | 5′ GCATACTTAAAACGCACTCG 3′ |
| Exon 5: | 5′ CCGCTCATAGCTTCTGTATG 3′ |
| | 5′ AACAATTCTCACATGCAAGC 3′ |
| Exons 6 and 7: | 5′ AGACTGACTTCAATAATATCC 3′ |
| | 5′ TCATTTGTTTCCCTAACCTAC 3′ |
| Exon 8: | 5′ ATTGATAGCTTATCGTTATGC 3′ |
| | 5′ AATGCTGAACATGAACTCTG 3′ |
| Exon 9: | 5′ ATGATAAAGCTCTTGGTATTTC 3′ |
| | 5′ TGCAGCATCATACGATCATG 3′ |
| Exon 10: | 5′ AATTCCAAGTGTAACTTCTG 3′ |
| | 5′ TAAAAGGTCCAAGTACGATC 3′ |
| Exon 11: | 5′ ACTGTCTCCATTCCTTTCAG 3′ |
| | 5′ ACCTTATGATCATGGTGGTG 3′ |
| Exon 12: | 5′ GAGGAATCTTTCCCAGAGC 3′ |
| | 5′ ATTAGTAGCAGCTGATACAG 3′ |
| Exon 13: | 5′ TATTCCCAGTGCATTGGTAC 3′ |
| | 5′ GAAAGTCATAAAGCTGACAG 3′ |
| Exon 14: | 5′ CTAATGTTCTCTCATAAAGTG 3′ |
| | 5′ GAACTAATGGAACCATCAAC 3′ |
| Exon 15: | 5′ CAGTCAGAATTGCATATACC 3′ |
| | 5′ AAGAGAAGACTATGTTACCC 3′ |
| Exon 16: | 5′ TTGGAATTATCTGTACTGTAC 3′ |
| | 5′ AGCCTAATGCAAAAGACATC 3′ |
| Exon 17: | 5′ ACTCCTTTCTCGTCTCTTTG 3′ |
| | 5′ TAGAGGTTGAAAACCACCAG 3′ |

Analysis of X-inactivation

To evaluate the state of X-inactivation in the mother of proband P55, an assay described by Pegoraro et al. was used (24), with which the methylation status of the polymorphic CAG repeat in the androgen receptor gene is tested, using the methylation-sensitive restriction enzyme Hpall. The PCR products of digested and undigested DNA were electrophoresed on 6% polyacrylamide gel and silver stained. The intensity of the bands was measured using the Diversity Database program (BIO-RAD) and the values obtained were corrected for preferential amplification of an allele (24).

Assay of FACL4 Activity on Lymphoblastoid Cells and Leukocytes

To test the activity of FACL4 on whole cell lysates, the assay described by Malhotra et al. was used (28). Briefly, enzymatic activity was determined by measuring the formation of (1-¹⁴C)-arachidonyl-CoA from 1-¹⁴C-labeled arachidonic acid. 10⁸ or 10⁷ lymphoblastoid cells were lysed in a lysis volume of 2 ml or 200 μl, respectively, and 20 μl of cell lysate were used to determine protein quantity (BIO-RAD). Subsequently, cell lysates were incubated for 20 min in 0.15 ml of a standard reaction mixture containing 15 μmol TRIS/HCl, pH 8.0, 1 μmol ATP, 100 nmol CoA, 750 nmol dithiothreitol, 3 μmol MgCl₂ and 40 μl of a solution of 50 mM NaHCO3, 7.5 mM Triton X-100, 10 nmol arachidonic acid and 2×10⁵ d.p.m. of labeled arachidonic acid. The reaction was stopped with 2.25 ml of propan-2-ol:heptane: 2 M sulphuric acid (40:10:1), followed by 1.5 ml of heptane and 1 ml of water and vigorous shaking. After centrifugation (5 min at 2000 rpm), the upper layer was removed and the lower aqueous phase was washed three times with 2 ml of heptane. The radioactivity in the upper (heptane) and lower phases (aqueous) was determined by scintillation counting (Beckman). To determine enzyme activity, the total radioactivity (lower plus upper phase) and the percentage of this radioactivity in the lower phase were calculated. This percentage correspond to the percentage of arachidonic acid used for the reaction (10 nmol) which has been converted to arachidonyl-CoA. The values were corrected for protein quantity.

To perform the test on leukocytes, 10 ml of blood was diluted with one volume of phosphate buffered saline (PBS) or physiological solution, mixed and carefully layered on one volume of Ficoll solution (Ficoll 99 g/l; sodium chloride 12 mmol/l; sodium diatrizoate 0,16 mol/l). After centrifugation (40 min at 2000 rpm), the upper layer of plasma and platelets was removed and the intermediate layer containing leukocytes was recovered to a fresh tube and washed twice with PBS or physiological solution. In order to eliminate the residual erythrocytes present after the treatment with ficoll, the pellet of leukocytes was resuspended in 1 ml of water, incubated in ice for one minute, diluted to 10 ml with PBS or physiological solution and centrifuged at 2000 rpm for 10 minutes. Leukocytes were also isolated from blood samples conserved at room temperature for 24, 72 and 120 hours. Isolated leukocytes were used immediately or cryopreserved at −80° C. until the test was performed. Both cryopreserved and room-temperature conserved leukocytes were lysed in 200 μl of lysis buffer and subjected to the enzymatic test using the protocol described above.

Anti-FACL4 Antibody

A polyclonal anti-FACL4 antibody was raised in rabbit with the synthetic peptide “KAKPTSDKPGSPYRS”, corresponding to a highly immunogenic coiled amino-terminal fragment of human FACL4 protein. The antibody was purified by affinity and used as the primary antibody (dilution 1:2000). In an immunoblotting assay, this antibody recognizes a protein of the expected size, absent in the liver. Since all members of the FACL family, except FACL4, are expressed in the liver, this assay demonstrates the specificity of the antibody for FACL4.

Immunohistochemical Staining

Staining was performed using immunoperoxidase on paraffin-embedded sections from hippocampus and cerebellum of normal adult subjects (obtained from the Pathology Services of the University of Siena). The 5-μm thick sections were deparaffinized and rehydrated. Endogenous peroxidase was stopped by incubation in 3% H₂O₂/methanol for 10 min.; the sections were pre-incubated in 1.5% BSA/PBS for 1 h RT; incubation in anti-FACL4 1:200 in PBS-BSA was performed overnight at 4° C. The secondary antibody conjugated with HRP (SIGMA-ALDRICH), diluted 1:200 in PBS-BSA, was incubated for 1 h RT. 3,3-diaminobenzidine tetrahydrochloride (SIGMA-ALDRICH) was used as a chromogen; the sections were counterstained with Mayer's haematoxylin, dehydrated and mounted with Histomount. The negative control slides were obtained by omitting the primary antibody. The slides were observed and photographed under a light microscope (DM Leitz).

Results

The authors have previously described a complex syndrome with contiguous genes deletion characterized by Alport syndrome, midface hypoplasia, mental retardation and elliptocytosis (AMME, OMIM #300194) due to a deletion of 2 Mb in Xq22.3 (2, 6). After the identification of the first family, the authors then identified a second family with a smaller deletion of about 1 Mb that presented with Alport syndrome (ATS) and mental retardation, and proposed the name of ATS-MR (Alport syndrome and Mental Retardation). This syndrome adds to ATS-DL syndrome (Alport syndrome and diffuse leiomyomatosis). In both syndromes, the gene COL4A5, which is responsible for ATS, is implicated, but, whereas ATS-DL extends centromerically, ATS-MR extends telomerically with respect to the collagen gene. A comparative analysis of the deletion extension between patients with ATS-MR syndrome and those with isolated ATS allowed the authors to limit the critical region for mental retardation to approximately 380 Kb. This region contains four genes: FACL4, KCNE5, NXT2 and GUCY2F.

The authors performed mutation analysis of these four genes in patients with isolated MR. Direct sequencing was carried out in 12 patients from unrelated families in which segregated nonspecific MR, mapping to regions of the X chromosome encompassing Xq22,3 (from Xq2l to Xq26) (7, 8). In one patient (T22), a point mutation was found, c.1585 C >A in exon 15 of FACL4 gene, encoding for acyl-CoA synthetase type 4 for long-chain fatty acids (FIG. 1a) To determine the mutation frequency of the FACL4 gene in XLMR, the mutation analysis was then extended to 107 unrelated male patients with XLMR. In one patient, P55, a mutation was found in the 3′ splice site of intron 10 of FACL4 (c.1003-2A>G) (FIG. 1b). Both mutations were absent in 300 normal controls (600 chromosomes). Patient T22 belongs to a family previously published as MRX63. The MRX locus in this family was mapped between DXS990 and DXS1227 (Xq21.33Xq27.1 ) with a Zmax/theta0 of 2.14 to DXS1001 (8). The affected males showed a nonspecific, nonprogressive moderate-to-severe mental deficit, without seizures (Table 1). The female carriers showed highly variable cognitive abilities, ranging from moderate MR to normal intelligence (I.2, II.4, III.1, all with an IQ>75). The affected males and mentally retarded female carriers showed a particular cognitive phenotype not found either in non-retarded carriers or in a non-carrier female with a low IQ (56) but with good social adaptation (III4).This cognitive profile is characterized by (i) difficulty in visuo-spatial structuring and (ii) executive function deficiency with weak verbal fluency, motor impulsiveness and selective attention deficit. The neurologic examination was normal, showing only slightly altered reflexes (Table 1). Also, female carrier II.3, with moderate mental deficit, showed at repeated clinical examinations, neurological features suggestive of a progressive cerebellar degeneration that was not observed in other family members, irrespective of their carrier status. Magnetic resonance imaging (MRI) studies revealed substantial cerebellar atrophy. Particular features observed in several affected males or in female carriers, as well as in several healthy family members, comprised unilateral ptosis (III.2, III.4), marked nasal tip (I.2, II.2, II.3, III.1, III.4), digital loops (I.2, II.3, II.4, III.1, III.2, III.4). Testicular volume and weight in the affected males were normal, as was the morphology of the face, hands and feet.

Patient P55 belongs to a small unpublished family. The three affected males are 10, 7 and 5 years of age. In all three cases, pregnancy and delivery were normal, as was motor development. The children do not present dysmorphic features. A significant speech delay was noted early, which worsened with time. The youngest brother is currently able to say a few words, while the older brother began association of two words at 6 years of age. The neurological examination was normal in all three brothers, and no epileptic phenomena were present. Assessment of IQ was attempted but failed because of severe speech delay and difficulties in understanding instructions. MR was estimated as being severe. In the younger brother the first signs were noted at 18 months of age, with language delay. Hyperactivity was also noted at the same age. MRI was normal. During a recent clinical examination, the patient presented with hyperactivity, attention loss and inability to concentrate. These behavioural problems are not present in the other two affected brothers. The mother seems normal, but no accurate assessment of her IQ was performed.

In both families, the mutations co-segregate with the disease (FIGS. 2a and 2b). Mutation c.1585C>A in family MRX63 leads to the substitution of arginine 529 (R570 in brain-specific isoform) with a serine inside the 25-residue motif characterizing acyl-CoA synthetases (FACS), which is common to both eukaryotic and prokaryotic FACS. Arginine 529 corresponds to arginine 23 of the consensus (FIG. 3). A site-directed mutagenesis of the acyl-CoA synthetase of E. Coli showed that the substitution of the corresponding arginine (arginine 453) completely abrogates enzymatic activity (9). The mutation c.1003-2A>G identified in patient P55 reveals a cryptic splice site located 28 bp before the correct splice site (FIG. 4a). Mutated mRNA contains 28 additional nucleotides between exon 10 and exon 11, with an in-frame stop codon (FIG. 4b, 4c). This produces a prematurely truncated protein, with 6 incorrect amino acids after proline 334. The protein should lack the second luciferase domain (LR2), containing the catalytic domain with the domain characterizing the FACS.

All six female carriers of family MRX63 showed completely skewed X-inactivation in leukocytes (8). Likewise, three female carriers out of the three belonging to the two ATS-MR families showed completely skewed X-inactivation in leukocytes (3). The authors also tested the carrier mother of patient P55 and obtained the same results. In all informative cases the. skewed X-inactivation was in favour of the normal X. The finding of completely skewed X-inactivation in the two families with point mutations in FACL4 described here strongly suggests that the same gene that causes MR also confers a selective advantage in leukocytes. This is consistent with the anti-apoptotic role of FACL4 (10). The X-inactivation status tested in blood does not correlate with the clinical status of females, since at least one carrier is affected in family MRX63. This result did not come totally unexpected. There is increasing evidence that the neurocognitive phenotype is not well correlated with X-inactivation assayed in blood (e.g. Rett syndrome, 11, 12). An explanation for this could be that X-inactivation is measured in blood and its status may be different in the brain or might have been different at some critical point during development.

Acyl-CoA synthetases are a family of enzymes that catalyze the formation of acyl-CoA from fatty acids, ATP and coenzyme A. Since FACL4 is expressed in lymphocytes (5), the authors tested enzymatic activity in lymphoblastoid cell lines from probands T22 and P55, an affected male patient (#850) and a carrier female of an ATS-MR family with the genomic deletion and normal controls. Since FACL4 has a high substrate preference for arachidonic acid, the authors used this fatty acid as a substrate. The patient with ATS-MR deletion showed a reduction in synthetase activity of approximately 88% with respect to the normal controls (FIG. 4). The same large decrease in activity was observed also in probands T22 and P55 (80% and 86% reduction, respectively; FIG. 4). As expected, lymphoblastoid cells of the carrier female showed normal, activity, due to the completely skewed X-inactivation (FIG. 4).

Direct sequencing was then performed on eight families, two with a diagnosis of nonspecific XLMR (L22 and L46) and six with a diagnosis of syndromic X-linked mental retardation (L49, L56, K8435, K8045, K8610 and K8835), mapping in a large interval encompassing Xq22.3. In one of the eight patients, L46, a point mutation was found, c.1001 C>T, in exon 10 of FACL4. Patient L46 belongs to a family published as MRX68 (XLMR Genes Update Web Site: http://xlmr.interfree.it/XLMR/Tab5.html). The MRX locus in this family was mapped between DXS8020 and DXS1220 (Xq2l.33-Xq23). The mutation c.1001 C>T leads to the substitution of proline 334, with a leucine inside the first luciferase domain (LRI) of the protein. Proline 334 is conserved in all known human and mouse FACL proteins. The mutation causes the abrogation of a restriction site for Mspl. Restriction analysis showed that the mutation co-segregates with the diseases in the family and was not found in 50 normal controls (100 chromosomes). The analysis of enzymatic activity performed on the patient's lymphoblastoid cells showed also in this case a dramatic reduction of activity compared with controls, demonstrating that the mutation is pathogenic. Also in this family, carrier females present a completely skewed X-inactivation.

In humans, five forms of FACL have been identified. FACL4 encodes for a protein of 670 amino acids expressed in various tissues, except for liver, the principal tissue of action of both FACL1 and FACL2 (5). In the brain, it encodes a longer transcript that results from an alternative splicing that produces a brain-specific isoform containing 41 additional N-terminal hydrophobic amino acids (GenBank accession number: Y12777 for the ubiquitous form and Y13058 for the brain-specific form) (5). The putative location predicted by the PSORT program varies from cytosol (0.45 probability) to the membrane of the endoplasmic reticulum (0.82 probability), if the 41 amino acids are added to the protein.

To determine the expression pattern of normal FACL4 protein and to determine its subcellular location, the authors performed immunohistochemical studies on adult human brain using a polyclonal antibody for a synthetic peptide. FACL4 is highly expressed in the human brain, especially in the cerebellum and hippocampus, with a distribution very similar to that obtained in the mouse (13) (FIG. 6). Cells of the pyramidal layer of hippocampus show a strong cytoplasmic staining of the soma; also the thin cytoplasmic ring of the granular cells of the dentate gyrus is reactive. Strong cytoplasmic staining is also evident in the soma of Purkinje's cells and the granular cells of the cerebellum. The proximal dendritic region of Purkinje's cells is also immunoreactive. The results showed that FACL4 is expressed specifically in the neurons, since the glial cells are completely negative. Within the neurons, the location is in the soma and the proximal region of the dendrites. The protein seems distributed diffusely in the cytoplasm (the nuclei are always negative), with accumulation near the nuclear membrane. This particular distribution could be due to the presence of the 41 N-terminal amino acids that localize the enzyme in the outer nuclear membrane.

The enzymatic assay of FACL4 activity represented a good tool, not only to confirm a mutation, but also to replace molecular analysis as a screening method. However, in its original form, the assay was performed on lymphoblastoid cells, obtained with EBV transformation of blood leukocytes. In order to bypass the cell transformation step, the authors gradually reduced the number of cells used for the assay from 10⁸ to 10⁷. This number correspond to the mean amount of leukocytes present in 10 ml of blood. The results showed that 10⁷ cells are enough to detect arachidonyl-CoA synthetase activity and to clearly distinguish a FACL4 mutation (FIG. 7, columns 1-8). Moreover, enzymatic activity observed with 10⁷ lymphoblastoid cells was comparable to that observed with leukocytes isolated from 10 ml of blood (FIG. 7, columns 1-8 vs 9-15). In addition, authors tested whether blood may be cryopreserved or stored at room temperature for several days before performing the assay. Results indicated that there is no difference in activity after cryopreservation (FIG. 7 column 11) or after 24 or 72 hours at room temperature (FIG. 7, columns 12-13). However, authors observed a significant reduction in activity after 120 hours at room temperature. (FIG. 7, column 14). These results indicate that the test can be performed directly on leukocytes isolated from blood conserved at room temperature for up to 72 hours. Authors proposed the enzymatic assay of FACL4 activity for the rapid screening of mentally retarded males. With respect to standard molecular analysis, this approach is less laborious, much faster and less expensive. In addition, the assay of FACL4 activity will let to identify promoter/ intron mutations, which are missed by standard mutation analysis of coding regions, and to overcome interpretation uncertainty usually associated with missense changes.

A possible mechanism for which reduced production of arachidonyl-CoA causes MR may be related to its role in signal transduction carried out by ion fluxes regulation, for example, of Ca2+ ions. In skeletal muscle, the reduced action on Ca2+ release by the Ca2+ release channel sensitive to ryanodine in the longitudinal tubules and the terminal cisternae of the sarcoplasmatic reticulum could be responsible for neonatal and infantile hypotonia common to males with ATS-MR and males of family MRX63 (14). An alternative mechanism could be related to apoptosis (10). Over-expression of FACL4 in EcR293 cells protects against apoptosis induced by arachidonic acid. However, inhibition of FACL4 activity promotes apoptosis induced by arachidonic acid (10). The germline absence of FACL4 function could lead to precocious apoptosis in neurons and to altered brain development.

So far, nine genes have been associated with nonspecific X-linked MR (MRX) (1). One is the gene adjacent to the fragile X-E site (FRAXE) on Xq28, called FMR2, which encodes a nuclear protein that may be a transcriptional regulator. Three genes, oligophrenin-1 (OPHN1) on Xq12, PAK3 on Xq21.3-q24 and ARHGEF6 on Xq26, encode proteins involved in the Rho GTPase pathways, which mediates cytoskeletal organization, cell shape and motility, and could be responsible for axonal outgrowth and the shape and size of dendrites. GDI1 in Xq28 is involved in synaptic vesicle cycling and neurotransmitter release. TM4SF2 (alias MSX1), in Xp11.4, interacts with beta-1 integrins and could play a role in the control of neurite outgrowth. IL1RAPL1, in Xp22.1-XP21.3, is homologous to the accessory proteins of the interleukin-1 receptor. Lastly, two genes associated with Coffin-Lowry and Rett syndromes are also involved in nonspecific MR: RPS6KA3 (RSK2) and MECP2, involved in the signal pathway of MAP kinase and in gene silencing, respectively (15-17). FACL4 is the tenth gene mutated in nonspecific X-linked MR (MRX) and is the first involved in fatty acid metabolism. FACL4 mutations could account for about 1 % of male nonspecific X-linked mental retardation A normal lipid homeostasis would therefore be critical for the correct development and/or functioning of the central nervous system.

Diagnostic Kit for MRX Syndrome

Kit for the Functional Assay (the Method is Described in the Materials and Methods Section)

Solution 1 (lysis buffer): 20 mM Tris-HCl pH 7.5; 140 mM sodium chloride; 5 mM EDTA; 1 mM magnesium chloride; IOmM sodium pyrophosphate;

NP-40

Leupeptin;

Phenylmethylsulfonyl fluoride (PMSF)

Coenzyme A;

Adenosine 5′ triphosphate (ATP);

Dithiothreitol;

Magnesium chloride;

100 mM Tris-HCI pH 8.

Solution 2: 50 mM sodium bicarbonate; 7.5 mM Triton X-100;

Cold arachidonic acid;

¹⁴C-labeled arachidonic acid.

Solution 3: 2-propanol/n-heptane/sulphuric acid (40:10:1);

n-heptane.

Kit to Reveal Mutations by SSCP or Direct Sequencing (the Method is Described in the Materials and Methods Section)

1. For the PCR phase:

Thermostable Taq polymerase;

Magnesium chloride;

Polymerase specific buffer;

Deoxynucleotides triphosphate;

Specific primers (sequences on pp. 8-9);

Control DNA from a subject not affected by XLMR;

Agarose to visualize amplification products.

b 2. For SSCP:

Polyacrylamide gradient gel (Pharmacia-Biotech) for GenePhor apparatus;

Silver staining reagents: absolute ethanol; nitric acid; silver nitrate; sodium carbonate; Formaldehyde; acetic acid.

3. For direct sequencing:

Oligonucleotides (the same used for PCR);

Kit for direct sequencing (BigDye Terminator Cycle Sequencing [Applied Biosystems]).

TABLE 1
Diagnostic Data of Family MRX63

| | | | | Deficit in | | | | | | | |
|---|---|---|---|---|---|---|---|---|---|---|---|
| | | Level | Lan- | visuo-spatial | | | | | | | |
| | | of au- | guage | structuring/ | | | | | | | |
| Patient | | ton- | impair- | executive | Behavior/ | Neurologic | head | Kyphosis/ | Infantile | | |
| (sex) | IQ | omy | ment | function | mood | at signs | circumference | scoliosis | hypotonia | MRI | Elliptocytosis |
| I2 (M) | <40 | mild | − | + | − | ↑ reflexes | −1.2SD | + | + | n..t. | n..t. |
| II2 (M) | 50 | Low | + | + | Marked | ↓ reflexes | −2SD | + | + | Normal | None |
| | | | | | anxiety | | | | | | |
| II3 (F) | 46 | mild | + | + | Depression | ↑ reflexes | Normal | − | − | Cerebral | n..t. |
| | | | | | | | | | | atrophy | |
| III2 (F) | 48 | Low | + | + | Marked | ↓ reflexes | −4SD | − | − | Normal | None |
| | | | | | anxiety | | | | | | |
| IV1 (M) | 37 | Low | − | n.t. | Autistic | ↑ reflexes | Normal | − | − | n..t. | n..t. |

n..t. = not tested

### REFERENCES

1. Chiurazzi, P., Hamel, B. C. J. & Neri, G. XLMR genes: update 2000. Eur J Hum Genet 9, 71-81 (2001 ).

2. Jonsson, J. et al. Alport syndrome, mental retardation, midface hypoplasia, and elliptocytosis: a new X-linked contiguous gene deletion syndrome? J Med Genet 35, 273-278 (1998).

3. Meloni, I. et al. Alport syndrome and mental retardation: clinical and genetic dissection of the contiguous gene deletion syndrome in Xq22.3 (ATS-MR). J Med Genet in press (2001).

4. Piccini, M. et a/. KCNE1 like gene is deleted in AMME contiguous gene syndrome: identification and characterisation of the human and mouse homologous. Genomics 60, 251-257 (1999).

5. Piccini, M. et al. FACL4, a new gene encoding Long Chain Acyl-CoA Synthetase 4, is deleted in a family with Alport syndrome, elliptocytosis and mental retardation. Genomics 47, 350-358 (1998).

6. Vitelli, F. et al. Identification and characterization of a higly conserved protein absent in the Alport S. (A), Mental retardation (M), Midface hypoplasia (M), and Elliptocytosis (E), contiguous gene deletion syndrome. Genomics 55, 335-340 (1999).

7. des Portes, V. et al. X-linked nonspecific mental retardation (MRX) linkage studies in 25 unrelated families: the European XLMR consortium. Am J Med Genet 85, 263-265 (1999).

8. Raynaud, M. et al. Systematic analysis of X-inactivation in 19 XLMR families: extremely skewed profiles in carriers in three families. Eur J Hum Genet 8, 253-258 (2000).

9. Black, P., Zhang, Q., Weimar, J. & DiRusso, C. Mutational analysis of a fatty Acyl-Coenzyme A Synthetase signature motif identifies seven amino acid residues that modulate fatty acid substrate specificity. Biol Chem 272, 4896-4903 (1997).

10. Cao, Y., Pearman, A. T., Zimmerman, G. A., Mcintyre, T. M. & Prescott, S. M. Intracellular unesterified arachidonic acid signals apoptosis. Proc Natl Acad Sci 97, 11280-11285 (2000).

11. Zappella, M., Meloni, I., Longo, I., Hayek, G. & Renieri, A. Preserved Speech Variants of the Rett Syndrome: Molecular and Clinical Dissection. Am J Med Genet 104, 14-22 (2001).

12. Nielsen, J. B. et al. MECP2 mutations in Danish patients with Rett syndrome: High frequency of mutations but no consistent correlations with clinical severity or with the X chromosome inactivation pattern. Eur J Hum Genet 9, 178-184 (2001).

13. Cao, Y., Murphy, K. J., McIntyre, T. M., Zimmerman, G. A. & Prescott, S. M. Expression of fatty acid-CoA ligase 4 during development and in brain. FEBS letters 467, 263-267 (2000).

14. Faergeman, N. J. & Knudsen, J. Role of long-chain fatty acyl-CoA esters in the regulation of metabolism and in cell signaling. Biochem J 323, 1-12 (1997).

15. Merienne, K. et al. A missense mutation in RPS6KA3 (RSK2) responsible for non-specific mental retardation. Nature Genet 22, 13-14,(L) (1999).

16. Meloni, I. et al. A mutation in the Rett syndrome gene, MECP2, causes X-linked mental retardation and progressive spasticity in males. Am J Hum Genet 67, 982-985 (2000).

17. Couvert, P. et al. MECP2 is highly mutated in X-linked mental retardation. Hum Mol Genet 10, 941-946 (2001).

18. Burgemeister, B., L., H.-B. & Lorge, I. Manual for the use of the Columbia Mental Maturity Scale (The Psychological Corporation, New York, 1954).

19. Goodglass, H. & Kaplan, E. Boston Diagnostic Aphasia Examination (Psychological Assessment Resources, Odessa, 1983).

20. McCarthy, D. Manual for the McCarthy scales of children's abilities (S.L.: The Psychological Corporation, New York, 1972).

21. Wechsler, D. Wechsler Memory Scale-Revised Manual. (The Psychological Corporation, New York, 1987).

22. Gauthier, L., Dehaut, F. & Joanette, Y. The Bell test: A quantitative and qualitative test for visual neglect. Int J Clin Neuropsychol 11, 49-54 (1989).

23. Association, A. P. Diagnostic and statistical manual of mental disorders. DSM-IV (American Psychiatric Association, Washington (DC), 1994).

24. Pegoraro, E. et al. Detection of new paternal dystrophin gene mutations in isolated cases of dystrophinopathy in females. Am J Hum Genet 54, 989-1003 (1994).

25. Stuhlsatz-Krouper, S. M., Bennett, N. E. & Schaffer, J. E. Substitution of alanine for serine 250 in the murine fatty acid transport protein inhibits long chain fatty acid transport. J Biol Chem 273, 28642-50 (1998).

26. Tanaka, T., Hosaka, K. & Numa, S. Long-chain acyl-CoA synthetase from rat liver. Methods in Enzymology 71, 334-41 (1981).

27. Watkins, P. A. et al. Disruption of the Saccharomyces cerevisiae FAT1 gene decreases very long-chain fatty acyl-CoA synthetase activity and elevates intracellular very long-chain fatty acid concentrations. J Biol Chem 273, 18210-9 (1998).

28. Malhotra, K. T., Malhotra, K., Lubin, B. H. & Kuypers, F. A. Identification and molecular characterization of acyl-CoA synthetase in human erythrocytes and erythroid precursors. Biochem J 344, 135-143 (1999).

29. Sambrook J, Fritsch E F, Maniatis T: Molecular Cloning, ed. 2. Cold Spring Harbour, Cold Spring Harbour Laboratory Press, 1989.

1. A nucleic acid molecule comprising at least one fragment of the human FACL4 gene that encodes for a functional portion of the FACL4 protein to be used in the diagnosis of MR-associated syndromes.

2. A nucleic acid molecule comprising at least one fragment of the human FACL4 gene that encodes for a functional portion of the FACL4 protein to be used in the therapy of MR-associated syndromes.

3. A method to detect in a subject at least one mutation of the gene encoding for the human FACL4 protein, located on the X chromosome, comprising the phases of:
a) collecting a specimen containing a sufficient quantity of the subject's DNA or able to be reproduced in culture;
b) isolating the DNA from the sample;
c) exponentially amplifying the DNA using as primer pair for the amplification reaction at least two oligonucleotides able to amplify a fragment of the human FACL4 gene, in which the fragment encodes for a functional portion of FACL4 protein;
d) detecting in at least one amplified fragment any mutations compared with a healthy control.

4. A method according to claim 3 in which the exponential DNA amplification phase is performed using primer pairs for the amplification reaction able to amplify the entire coding portion of the human FACL4 gene.

5. A method according to claim 4 in which the exponential DNA amplification phase to amplify the entire coding portion of the human FACL4 gene will comprise the use of the following primer pairs:

| | |
|---|---|
| Exon 3: | 5′ GTGAGCACATTTAGCTTAAG 3′, |
| | 5′ ATCAATTGTGCTATCAACTTG 3′; |
| Exons 3 and 4: | 5′ CTTCTTCAGCACAATAAGGC 3′, |
| | 5′ GCATACTTAAAACGCACTCG 3′; |
| Exon 5: | 5′ CCGGTCATAGCTTCTGTATG 3′, |
| | 5′ AACAATTCTCACATGCAAGC 3′; |
| Exons 6 and 7: | 5′ AGAGTGACTTCAATAATATCC 3′, |
| | 5′ TCATTTGTTTCCCTAACCTAC 3′; |
| Exon 8: | 5′ ATTGATAGCTTATCGTTATGC 3′, |
| | 5′ AATGCTGAACATGAACTCTG 3′; |
| Exon 9: | 5′ ATGATAAAGCTCTTGGTATTTC 3′, |
| | 5′ TGCAGCATCATACGATCATG 3′; |
| Exon 10: | 5′ AATTCCAAGTGTAACTTCTG 3′, |
| | 5′ TAAAAGGTCCAAGTACGATC 3′; |
| Exon 11: | 5′ ACTGTCTCCATTCCTTTCAG 3′, |
| | 5′ ACCTTATGATCATGGTGGTG 3′; |
| Exon 12: | 5′ GAGGAATCTTTCCCAGAGC 3′, |
| | 5′ ATTAGTAGCAGCTGATACAG 3′; |
| Exon 13: | 5′ TATTCCCAGTGCATTGGTAC 3′, |
| | 5′ GAAAGTCATAAAGCTGACAG 3′; |
| Exon 14: | 5′ CTAATGTTCTCTCATAAAGTG 3′, |
| | 5′ GAACTAATGGAACCATCAAC 3′; |
| Exon 15: | 5′ CAGTCAGAATTGCATATACC 3′, |
| | 5′ AAGAGAAGACTATGTTACCC 3′; |
| Exon 16: | 5′ TTGGAATTATCTGTACTGTAC 3′, |
| | 5′ AGCCTAATGCAAAAGACATC 3′; |
| Exon 17: | 5′ ACTCCTTTCTCGTCTCTTTC 3′, |
| | 5′ TAGAGGTTGAAAACCACCAG 3′. |

6. A method according to claims 3 to 5 in which the phase of demonstrating, in at least one amplified fragment, mutations compared with a healthy control will be done by direct sequencing or the SSCP method.

7. A diagnostic kit for MR-associated syndromes, using the method according to claims 3 to 6, comprising:
a) at least one pair of primer oligonucleotides for the exponential amplification reaction of at least one fragment of the human FACL4 gene, in which the fragment encodes for a functional portion of the FACL4 protein;
b) a control DNA from a subject not affected by XLMR.

8. A kit according to claim 7 in which the oligonucleotide primer pairs for the amplification reaction are able to amplify the entire region coding for the FACL4 gene.

9. FACL4 protein or a functional portion thereof for the diagnosis of MR-associated syndromes.

10. FACL4 protein or a functional portion thereof for the therapy of MR-associated syndromes.

11. A method for the determination of the enzymatic activity of FACL4 protein in a biologic sample, comprising the phases of:
a) collecting a biological sample from the subject, in which the sample is comprised in the group of biological fluids, lysed lymphoblastoid cells, leukocytes;
b) incubating the sample in an appropriate reaction mixture containing arachidonic acid;
c) detecting arachidonyl-CoA production.

12. A method according to claim 11 in which the detection of arachidonyl-CoA is performed using labeled arachidonic acid.

13. A diagnostic kit for MR-associated syndromes to work the method according to claims 11 or 12, comprising:
a) Lysis buffer, with appropriate protease inhibitors and/or reduction agents;
b) Coenzyme agent A and Adenosine 5′triphosphate (ATP);
c) Cold arachidonic acid and ¹⁴C-labeled arachidonic acid.


SEQUENCE LISTING (33 sequences)

SEQ ID NO: 1 - DNA, 20 bp, artificial sequence
  primer
gtgagcacatttagcttaag

SEQ ID NO: 2 - DNA, 21 bp, artificial sequence
  primer
atcaattgtgctatcaacttg

SEQ ID NO: 3 - DNA, 20 bp, artificial sequence
  primer
cttcttcagcacaataaggc

SEQ ID NO: 4 - DNA, 20 bp, artificial sequence
  primer
gcatacttaaaacgcactcg

SEQ ID NO: 5 - DNA, 20 bp, artificial sequence
  primer
ccgctcatagcttctgtatg

SEQ ID NO: 6 - DNA, 20 bp, artificial sequence
  primer
aacaattctcacatgcaagc

SEQ ID NO: 7 - DNA, 21 bp, artificial sequence
  primer
agactgacttcaataatatcc

SEQ ID NO: 8 - DNA, 21 bp, artificial sequence
  primer
tcatttgtttccctaacctac

SEQ ID NO: 9 - DNA, 21 bp, artificial sequence
  primer
attgatagcttatcgttatgc

SEQ ID NO: 10 - DNA, 20 bp, artificial sequence
  primer
aatgctgaacatgaactctg

SEQ ID NO: 11 - DNA, 22 bp, artificial sequence
  primer
atgataaagctcttggtatttc

SEQ ID NO: 12 - DNA, 20 bp, artificial sequence
  primer
tgcagcatcatacgatcatg

SEQ ID NO: 13 - DNA, 20 bp, artificial sequence
  primer
aattccaagtgtaacttctg

SEQ ID NO: 14 - DNA, 20 bp, artificial sequence
  primer
taaaaggtccaagtacgatc

SEQ ID NO: 15 - DNA, 20 bp, artificial sequence
  primer
actgtctccattcctttcag

SEQ ID NO: 16 - DNA, 20 bp, artificial sequence
  primer
accttatgatcatggtggtg

SEQ ID NO: 17 - DNA, 19 bp, artificial sequence
  primer
gaggaatctttcccagagc

SEQ ID NO: 18 - DNA, 20 bp, artificial sequence
  primer
attagtagcagctgatacag

SEQ ID NO: 19 - DNA, 20 bp, artificial sequence
  primer
tattcccagtgcattggtac

SEQ ID NO: 20 - DNA, 20 bp, artificial sequence
  primer
gaaagtcataaagctgacag

SEQ ID NO: 21 - DNA, 21 bp, artificial sequence
  primer
ctaatgttctctcataaagtg

SEQ ID NO: 22 - DNA, 20 bp, artificial sequence
  primer
gaactaatggaaccatcaac

SEQ ID NO: 23 - DNA, 20 bp, artificial sequence
  primer
cagtcagaattgcatatacc

SEQ ID NO: 24 - DNA, 20 bp, artificial sequence
  primer
aagagaagactatgttaccc

SEQ ID NO: 25 - DNA, 21 bp, artificial sequence
  primer
ttggaattatctgtactgtac

SEQ ID NO: 26 - DNA, 20 bp, artificial sequence
  primer
agcctaatgcaaaagacatc

SEQ ID NO: 27 - DNA, 20 bp, artificial sequence
  primer
actcctttctcgtctctttc

SEQ ID NO: 28 - DNA, 20 bp, artificial sequence
  primer
tagaggttgaaaaccaccag

SEQ ID NO: 29 - DNA, 20 bp, artificial sequence
  primer
atgaatcggtgtgtctgagg

SEQ ID NO: 30 - DNA, 21 bp, artificial sequence
  primer
atcccatggagatgttctgtc

SEQ ID NO: 31 - DNA, 19 bp, artificial sequence
  primer
ggaagcaaaggaactgtac

SEQ ID NO: 32 - DNA, 20 bp, artificial sequence
  primer
atgaatcggtgtgtctgagg

SEQ ID NO: 33 - protein, 15 aa, artificial sequence
  synthetic peptide
KAKPTSDKPGSPYRS